(19) United States
(12) Patent Application Publication
(10) Pub. No.: US 2022/0270727 A1
(43) Pub. Date: Aug. 25, 2022

(54) METHOD AND APPARATUS FOR GENERATING AN ELECTRONIC MEDICAL BADGE

(71) Applicant: SecondOpinionExpert, Inc., Westlake Village, CA (US)

(72) Inventor: Mohan Ananda, Westlake Village, CA (US)

(21) Appl. No.: 17/180,863

(22) Filed: Feb. 22, 2021

(51) Int. Cl. G16H 10/65; G06K 19/06; G06K 7/14; G16H 15/00

(52) U.S. Cl. CPC G16H 10/65; G06K 19/06037; G06K 7/1417; G16H 15/00; A61B 90/96

(57) ABSTRACT

A system is disclosed to provide a user the ability to generate an electronic medical badge whereby the electronic medical badge includes medical vaccine information relating to the user. The system enables the user to identify a medical vaccine that the user recently obtained, and provide information including the vaccine provider, the vaccine manufacturer, the vaccine dose, and the vaccine location. The system then generates an electronic medical badge comprising a 2D barcode that may be displayed on a smart device. The system stores the electronic medical badge in a user's personal health digital records vault which may be stored on the user's smart device. The system may also generate a personal health digital records vault report wherein the user's electronic medical badge is included in the personal health digital records vault report.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

### Field

This disclosure relates to the field of using technology to create an electronic medical badge to allow entities to verify a user's medical vaccination history and medical testing history related to infectious diseases and viruses transmitting through a community. The system described herein uses a software architecture that ensures secure data communications for receiving and analyzing data related to a user's vaccination and testing history and the system then generates an electronic medical badge for a user to display for entities to verify a user's vaccination and testing information. The system also generates a personal health digital records vault which stores a user's medical vaccine and medical testing information.

### Description of the Related Art

New viruses and infectious diseases can spread rapidly through communities infecting hundreds of thousands of individuals at a time and crippling the economic and health systems of cities, states, and countries. As seen recently with the recent COVID-19 virus which resulted in a global pandemic due to the ease of transmission, it is more and more essential for individuals to be able to demonstrate to employers and other authorities that an individual is vaccinated against a virus or has tested negative for a certain virus.

Employers may wish to know which of their employees has been vaccinated before an employee is permitted to enter an office. Similarly, travel agencies may wish to verify a traveler has obtained a vaccine, or at least has tested negative for a certain vaccine, before permitted the traveler to enter a bus, train, vehicle, or board a plane. In addition, large venues such as professional sporting events, concerts, trade shows and conferences, may wish to confirm that all those attending the event have been vaccinated or have tested vaccine for any new virus in the community.

Therefore, there is now a pressing need for individuals to provide verification to others of their medical vaccination history or medical testing history. Many computer systems exist to store medical information for an individual. However, systems do not exist that allow a user to easily access their medical vaccination history or medical testing history. For example, if a user wishes to know when they last received a tetanus shot, then they will likely have to consult their physician. At present, there are not any systems which allow a user to easily access of all their medical vaccination history or medical testing history. Therefore, there exists a need for a user to be able to easily access any of their medical vaccination history or medical testing history so that they can have others verify their medical vaccination status or medical testing status and therefore allow the user to partake in various commitments.

### DESCRIPTION OF THE DRAWINGS

FIG. 1 is a block diagram of an environment for a system for generating an electronic medical badge.

FIG. 2 is a block diagram of a system for generating an electronic medical badge.

FIG. 3 is a flowchart for generating an electronic medical badge and a personal health digital records vault report.

FIG. 4 is a flowchart for the initiation of generating an electronic medical badge.

FIG. 5 is a flowchart of for generating an electronic medical badge.

FIG. 6 is a flowchart for uploading digital medical records to be used to generate an electronic medical badge.

FIG. 7 is a flowchart for faxing medical records to be used to generate an electronic medical badge.

FIG. 8 is a flowchart for generating an electronic medical badge and embedding it in a report.

FIG. 9 is a flowchart for scanning an electronic medical badge.

FIG. 10 is an example of a comprehensive user's personal health digital records vault report.

FIG. 11 is an example of a portion of a user's personal health digital records vault report that may be displayed on a user's electronic device.

FIG. 12 is an example of another user's display after scanning the user's electronic medical badge.

FIG. 13 is an example of a portion of a user's personal health digital records vault report that may be displayed on a user's electronic device showing a user's vaccination information.

FIG. 14 is another example of another user's display after scanning the user's electronic medical badge.

Throughout this description, elements appearing in figures are assigned three-digit or four-digit reference designators, where the most significant digit is the figure number and the two least significant digits are specific to the element. An element that is not described in conjunction with a figure may be presumed to have the same characteristics and function as a previously described element having a reference designator with the same least significant digits.

### DETAILED DESCRIPTION

### Description of Apparatus

For purposes of this patent, the term “user” as used herein means an individual who has either taken a medical test, or obtained a medical vaccine, for a particular infectious disease or virus. For purposes of this patent, the term “vaccine provider” means the entity who is administering the vaccine for the user. The vaccine provider may be the manufacturer of the vaccine, or it may be a different entity that is administering a vaccine manufactured by another company.

Described herein is a system for generating an electronic medical badge for a user. One of the primary objectives of the system is to leverage technology to assist an entity to verify that an individual has obtained a medical vaccine or a medical test to verify the user does not have a certain infectious disease or virus. The system for generating an electronic medical badge may be used by employers, travel agencies, organizers of large venues, governmental agencies, and other companies to determine whether to allow a certain user to use certain facilities or attend certain gatherings. For example, employers may wish to confirm their employees have received a vaccine before allowing the employees to physically enter the work environment. Travel agencies, airlines, and airports may wish to ensure any travelers similarly have received a vaccine before allowing the travelers to board a bus, vehicle, train, ship, or an airplane. Similarly, concert halls and stadiums may wish to ensure that anyone entering their venues has been vaccinated to minimize any risk of allowing a virus to spread rapidly in their venue.

Similarly, a user may wish to use the system to inform others that he or she has taken a medical test which establishes that the user is not carrying the virus to ensure that they will not spread the virus to others in proximity. For example, for users who have not had the chance to obtain a vaccine, these users may be able to take a medical test to show a negative result to allow them to travel on a plane or attend a concert.

A user's vaccine information, or medical test information, can be stored in a computerized system. This information can then be used to generate an electronic medical badge. The electronic medical badge may be a 2D barcode that a user can display on a smart device which when scanned will inform an entity that this user has either tested negative for the virus, or this user has been vaccinated. The 2D barcode will include information as to the vaccine provider who administered the vaccine, the vaccine manufacturer, if different than the vaccine provider, the date the vaccine was administered, the location the vaccine was administered, and the dose that was administered. For example, if a vaccine requires multiple doses for a user, then the 2D barcode will identify how many of the doses have been administered, and the date of those vaccines. In addition, the system may generate reminders for the user to schedule additional doses required to complete all the doses of the vaccine.

Similarly, if a user has not yet been vaccinated, but has taken medical tests, then the 2D barcode will identify the type of medical test that was taken, the date the test was taken, and the result of the test. For example, the types of medical tests available to detect the COVID-19 virus include a molecular test, such as RT-PCR tests, and antigen tests that detect specific proteins from the virus. The 2D barcode may identify which type of test the user has taken, the date the test was taken, and the result of the test.

The 2D barcode may be a QR code, or it may be a proprietary barcode. The 2D barcode may be scanned using a camera on a smart device, or a 2D barcode scanner, and after it is scanned, it will trigger an inquiry to the system to return and display the information stored in the system, in which the information displayed is based on the contents of the original document stored on the system and which the system has verified the contents of the document.

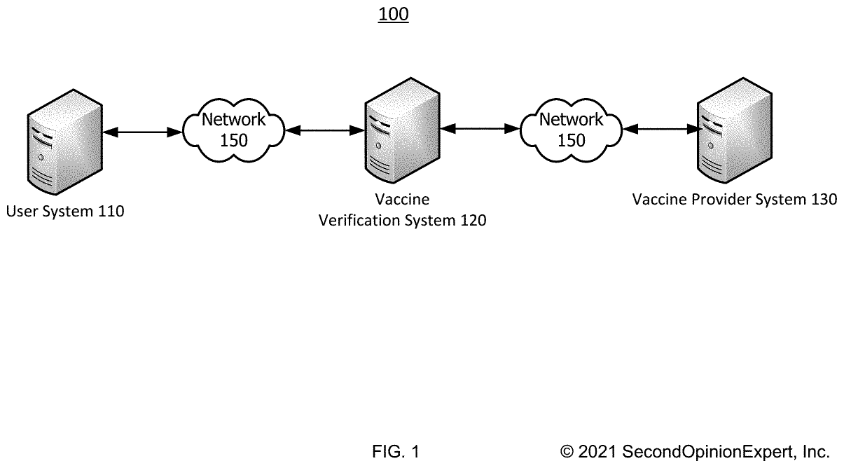

A user may use the system shown in FIG. 1 to generate an electronic medical badge. Referring now to FIG. 1, there is shown an environment 100 for a system for generating an electronic medical badge. The environment 100 includes user system 110, vaccine verification system 120 and vaccine provider system 130, all of which are interconnected via network 150. The network 150 may be a Local Area Network (LAN), a Wide Area Network (WAN), a Storage Area Network (SAN), wired, wireless, or a combination of these, and may include or be the Internet.

An electronic medical badge is a 2D barcode which provides information regarding the vaccine the user has received, or the results of any medical test the user has taken. The 2D barcode may also contain all vaccines the user has received throughout his whole life, or it may only identify the vaccine information for a particular virus. The electronic medical badge is generated by a vaccine verification system, such as vaccine verification system 120 in FIG. 1 and which is connected to a user system.

The user system 110 is connected to the network 150. The user system 110 is a computing device including software suitable for obtaining medical diagnostic data relating to a user's vaccination history and medical test history. The user system is associated with an individual user (not shown) and is capable of interfacing with the user to obtain personal information, medical information, historical information, diagnostic information, and vaccination information and medical testing information conducted on the user. The user system 110 may be a stand-alone computing device, a personal computer, lap-top computer, mobile device, smart watch, a tablet PC, or a smartphone.

Similarly, the vaccine verification system 120 is connected to the network 150. The vaccine verification system 120 is a computing device including software suitable for obtaining medical diagnostic data relating to a user's medical vaccine information, or medical test information, storing medical information and generating an electronic medical badge for a user. The vaccine verification system 120 may be a stand-alone computing device, a personal computer, lap-top computer, mobile device, smart watch, a tablet PC, a personal digital assistant, a smartphone, a server computer operating as a part of a distributed or peer-to-peer network or many other forms, a notebook, or a mobile phone.

Finally, the vaccine provider system 130 is connected to the network 150. The vaccine provider system 130 is a computing device including software suitable for obtaining medical vaccination information, or medical testing information relating to a user's medical vaccination or testing history and transmitting personal health digital records reports to the vaccine verification system 120. The vaccine provider system is associated with a company (not shown) who administers the vaccine for the user. The vaccine provider system 130 may be a stand-alone computing device, a personal computer, lap-top computer, mobile device, smart watch, a tablet PC, a personal digital assistant, a smartphone, a server computer operating as a part of a distributed or peer-to-peer network or many other forms, a notebook, a netbook or a mobile phone, that is running software suitable for interfacing with a vaccine provider.

The network 150 may take the form of a local network, a wide area network, the Internet or any number of other networks. The network 150 may be implemented locally by physically connected computers or may be distributed over a wide area.

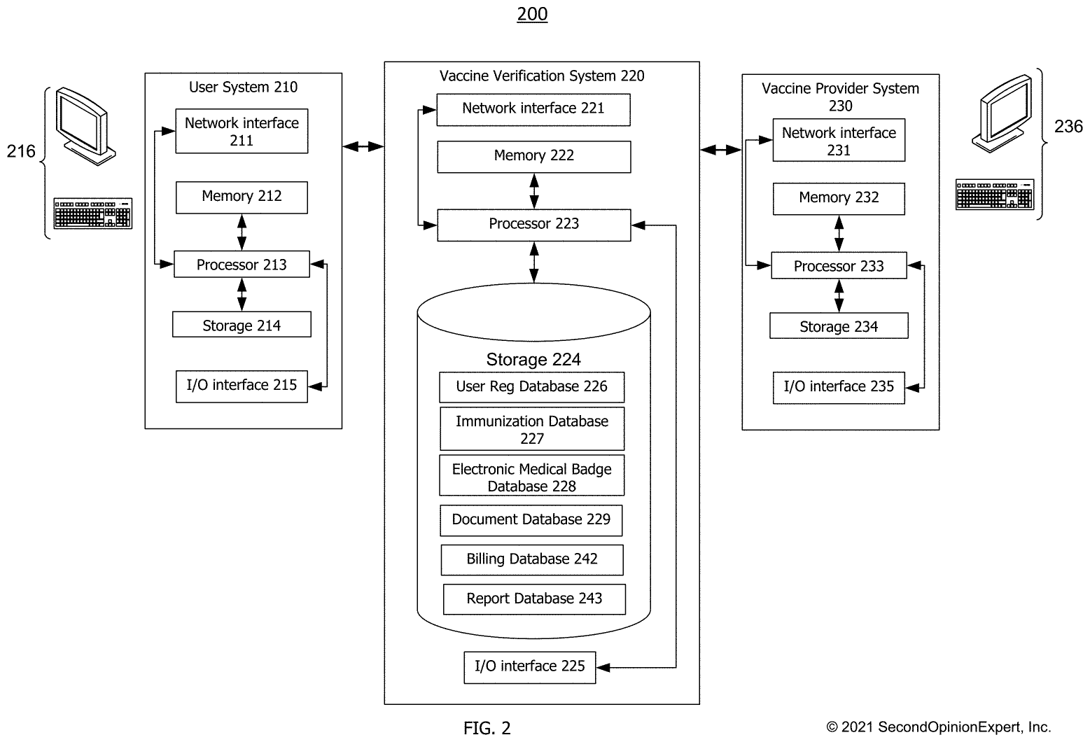

Referring now to FIG. 2, there is shown a block diagram of a system 200 for generating an electronic medical badge. As in FIG. 1, the system comprises a user system 210, a vaccine verification system 220 and a vaccine provider system 230 all interconnected by the network 250.

The user system 210 comprises a network interface 211, a memory 212, a processor 213, a storage 214 and an I/O interface 215. The processor 213 may be or include one or more microprocessors, application specific integrated circuits (ASICs), programmable logic devices (PLDs) and programmable logic arrays (PLAs).

The network interface 211 includes an interface to a network such as network 250 and network 150 in FIG. 1.

The memory 212 may be or include RAM, ROM, DRAM, SRAM and MRAM, and may include firmware, such as static data or fixed instructions, BIOS, system functions, configuration data, and other routines used during the operation of the user system 210 and processor 213. The memory 212 also provides a storage area for data and instructions associated with applications and data handled by the processor 213.

The I/O interface 215 interfaces the processor 213 to peripherals such as display and keyboard 216. The I/O interface also interfaces the processor 213 to peripherals (not shown) such as webcams, fax machines, printers, USB devices, microphones, and speakers.

The storage 214 provides non-volatile, bulk, or long-term storage of data or instructions in the user system 210. The storage 214 may take the form of a disk, tape, CD, DVD, or other reasonably high capacity addressable or serial storage medium. Multiple storage devices may be provided or available to the user system 210. Some of these storage devices may be external to the user system 210, such as network storage or cloud-based storage. As described herein, “storage” does not include transitory medium such as signals or electrical current. “Storage” as used herein is limited to non-transitory storage medium.

The vaccine verification system 220 comprises a network interface 221, a memory 222, a processor 223, and a storage 224. The processor 223 is similar to processor 213 and it may be or include one or more microprocessors, application specific integrated circuits (ASICs), programmable logic devices (PLDs) and programmable logic arrays (PLAs).

The network interface 221 includes an interface to a network such as network 250 and network 150 in FIG. 1. The network interface 221 is similar to network interface 211 in user system 210.

The memory 222 is similar to memory 212 in user system 210. The memory 222 212 may be or include RAM, ROM, DRAM, SRAM and MRAM, and may include firmware, such as static data or fixed instructions, BIOS, system functions, configuration data, and other routines used during the operation of the vaccine verification system 220 and processor 223. The memory 222 also provides a storage area for data and instructions associated with applications and data handled by the processor 223.

The I/O interface 225 interfaces with the processor 223 to peripherals, such as displays, keyboards, webcams, fax machines, printers, USB devices, microphones and speakers (not shown).

The storage 224 provides non-volatile, bulk or long-term storage of data or instructions in the vaccine verification system 220. The storage 224 may take the form of a disk, tape, CD, DVD, or other reasonably high capacity addressable or serial storage medium. Multiple storage devices may be provided or available to the vaccine verification system 220. Some of these storage devices may be external to the vaccine verification system 220, such as network storage or cloud-based storage. In this patent, the term “storage medium” does not encompass transient media such as signals and waveforms that convey, but do not store information.

The storage 224 may store data required for the system. For example, the storage may have a user registration database 226, an immunization database 227, an electronic medical badge database 228, a document database 229 and a medical billing database 242. The user registration database 226 may include information regarding a user's account information, his login information, and security questions that may be asked in the event the user requests a new password for his account. The immunization database 227 may include information regarding vaccination information for the user, and medical testing information relating to medical tests the user has taken to detect certain viruses. For example, the immunization database may include an entry for the COVID-19 virus, and the database may store information as to whether the user has received a vaccine for the COVID-19 virus, or if the user has taken a medical test to determine if he has the COVID-19 virus.

The electronic medical badge database 228 may store 2D barcodes that are generated with the vaccine information or the medical testing information for the user for a particular vaccine. For example, the electronic medical badge database may store a 2D barcode containing information about any vaccines the user has received for a particular virus, such as the COVID-19 virus. When this 2D barcode is scanned by someone, his device will display the contents of the vaccination or testing information for the user. Alternatively, the electronic medical badge database may store a 2D barcode that includes all of the vaccines the user has ever taken in his life. There may be instances, such as in an emergency, whereby medical professionals may wish to know all of the vaccines a certain user has taken. In those instances, the user may wish to have the electronic medical badge display all of the vaccines he has ever been administered so as to inform the medical professionals the medical vaccination history of the user. The medical billing database 242 may include information regarding the user's medical insurance information and payment plan.

The document database 229 is a database that may store documents relating to the user's medical vaccination history, or medical testing history. The document database may store links to locations on the system where the documents are stored. The document database 229 may also include a Dicom (Digital Imaging and Communications) database. A Dicom database is a database that stores digital images relating medical diagnostic test results. The Dicom standard is a standard for transferring images and associated information between devices. It provides a standard for how digital diagnostic images are to be transferred. The Dicom database stores the digital images in a manner that is compliant with the Dicom standard.

The report database 243 may store reports that are generated by the system for the user. For example, the system may store a current status report which is generated by the system. The current status report may include all of the medical testing information and the medical vaccination information for the user. For example, the current status report may include information regarding the type of medical test conducted, the results of the tested, and the location the test was administered. Similarly, the current status report may include information regarding any vaccines the user has been administered, the manufacturer of the vaccine, the provider of the vaccine, the location the vaccine was administered, the date the vaccine was administered, and the vaccine dose. The current status report may also include information about any upcoming vaccine appointments whereby a user may be administered additional doses of a particular vaccine. For example, multiple vaccines for the COVID-19 virus require multiple doses. The current status report may indicate if the user has an upcoming vaccine dose that is due, and how many doses remain for the vaccine.

It is to be understood that the databases shown in the storage 224, can be merged into one database or can exist in separate databases. The databases as shown in FIG. 2 is merely exemplary and it is not required that the system have this storage organization. Additional databases may also be used to store the data required by the system.

The vaccine provider system 230 comprises a network interface 231, a memory 232, a processor 233, a storage 234 and an I/O interface 235. The processor 233 may be or include one or more microprocessors, application specific integrated circuits (ASICs), programmable logic devices (PLDs) and programmable logic arrays (PLAs).

The network interface 231 includes an interface to a network such as network 250 and network 150 in FIG. 1.

The memory 232 may be or include RAM, ROM, DRAM, SRAM and MRAM, and may include firmware, such as static data or fixed instructions, BIOS, system functions, configuration data, and other routines used during the operation of the user system 210 and processor 213. The memory 232 also provides a storage area for data and instructions associated with applications and data handled by the processor 213.

The I/O interface 235 interfaces the processor 233 to peripherals such as display and keyboard 236. The I/O interface 235 also interfaces the processor 233 to peripherals (not shown) such as webcams, fax machines, printers, USB devices, microphones and speakers.

The storage 234 provides non-volatile, bulk or long term storage of data or instructions in the physician system 230. The storage 234 may take the form of a disk, tape, CD, DVD, or other reasonably high capacity addressable or serial storage medium. Multiple storage devices may be provided or available to the physician system 230. Some of these storage devices may be external to the physician system 230, such as network storage or cloud-based storage. In this patent, the term “storage medium” does not encompass transient media such as signals and waveforms that convey, but do not store information.

The user system 210 and the vaccine provider system 230 may also comprise a display and an input unit such as 216 and 236. The vaccine verification system 220 may also comprise a display and an input unit, although not shown in the figure. Further, the user system 210 and the vaccine provider system 230 may also comprise additional components such as printers, fax machines, scanners, microphones, speakers, and USB devices.

The system can connect users with the vaccine providers to ensure a user's medical history remains current with respect to the vaccination information and medical testing information for the user, while also including test results and images for any entities requiring verification that a user has been administered a certain vaccine. The system is a web-based HIPAA compliant and secure system that allows users a convenient way to register and upload their medical vaccination information, medical test results and diagnostic images. The system is designed to ensure compliance with the HIPAA encryption standards to ensure the security and privacy of an individual's Protected Health Information (PHI). The system will be updated to support new standards issued by the National Institute of Standards and Technology (NIST) regarding the HIPAA encryption standards.

The system may be integrated with various medical insurance carriers such that a user's bill may be sent to the appropriate carrier.

The system may follow up with users to inform the user about another vaccine dose that needs to be administered for the user.

Some of the benefits of the vaccine verification system include a verification of the vaccines and testing that a user has conducted to determine if a user is carrying a certain transmissible virus. Large venues and crowded locations need to quickly verify that those individuals entering their premises may not be carrying a highly transmissible virus, such as COVID-19. The vaccine verification system will enable entities to quickly determine if a user should be permitted to physically enter a building, board a plane, or attend a large professional sporting event in a large stadium. This can greatly reduce the spread of a virus within a community.

Description of Processes

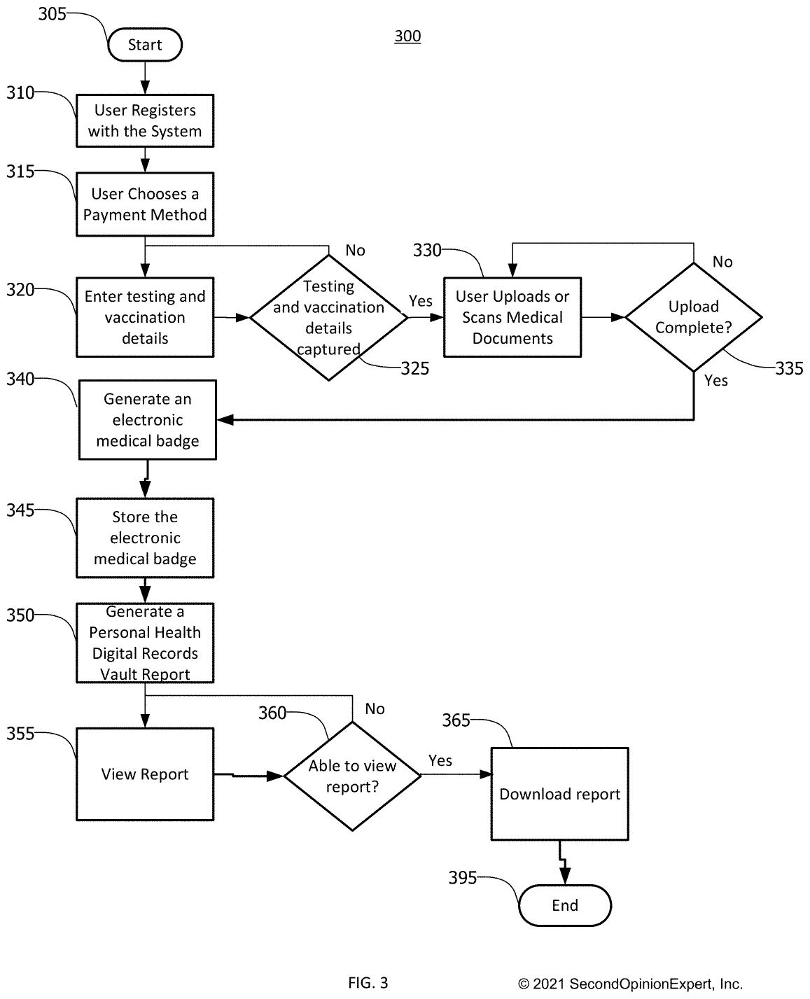

Referring now to FIG. 3, a process 300 for generating an electronic medical badge and a personal health digital records vault report is shown. The process 300 may start at 305 and may finish at 395. The process may be started by a user using a user system, such as user system 110 in FIG. 1.

The process 300 may begin with a user registering with the system at 310. A user registers with the system by entering personal information, such as his name, birth date, the location he resides, any pre-existing conditions, and gender. After entering personal information, the user may choose a username and password that is secure and unique to the user. After selecting a username and password, the user may login into the system to begin entering the information to generate an electronic medical badge.

After registering with the system, the user chooses a payment method at 315. At this step, the user enters his medical insurance information into the system so that the system can bill the appropriate insurance on behalf of the user. In addition, the user may enter a credit card, or bank account information, to keep on file in the event there are expenses that are not covered by the insurance that need to be paid by the user. Alternatively, the system may require the user to pay for the service upfront and then bill the insurance. In such an instance, if the insurance pays the bill, either in full or in part, the user will be credited the amount that the insurance paid. The system may store the user's medical insurance information as well as any credit card or bank account information in a database so that it will remain on file in the user's account.

After the user has entered the payment information at 315, the system starts requesting testing and vaccination information from the user at 320. For example, the user may enter the type of medical test he took, and the viruses or infectious diseases for which the test shows results. Similarly, the user may enter the vaccination that he was administered, and the provider that administered the vaccine, and the manufacturer of the vaccine. In addition, the user may identify the dose of the vaccine that he was administered. For example, some vaccines include multiple doses of the vaccine. For example, the Pfizer-BioNTech COVID-19 vaccine requires two doses that are administered 21 days apart from each other. Alternatively, the Moderna COVID-19 vaccine requires two doses that are administered 28 days apart from each other. Therefore, the user will enter what number dose he was administered, and if additional doses need to be obtained.

After the user enters the testing and vaccination information, the system reviews the information to determine if the testing and vaccination details were captured at 325. For example, the system may determine that the user entered that he took the Pfizer-BioNTech COVID-19 vaccine, but if the user did not identify which of the two doses he was administered, then the system may determine that the accurate vaccine details have not yet been captured. If the system makes the determination that additional information is required, then the process returns to 320 to allow the user to enter the additional testing and vaccination information. After the user enters the additional testing and vaccination information, then the system proceeds again to 325 to verify that the testing and vaccination details have been captured.

If the system concludes that all the information has been entered, then the system proceeds to 330 to allow the user to upload or scan the medical documents revealing the information. For example, if the user took an antigen test to determine if he had COVID-19, then the user might upload the document showing the lab test results. Alternatively, if the user was administered a vaccine, then the user may upload or scan the physical card that the user received when he was administered the vaccine. If the user has multiple test documents or vaccine documents, then the user may upload all of the documents to the system. The system may store images of the documents in the system. Alternatively, the system may store a link in the system which will direct the system to the document stored in the system. Before the system stores the document, the system verifies the validity of the document. For example, the system verifies that the data in the document (such as the vaccine provider, the vaccine dose, the date of the vaccine dose) is accurate. The system may verify this information by comparing the information entered by the user with information retrieved from the vaccine provider, or another third party storing the information. If the data in the document is not accurate, the system requests the user to re-enter the accurate information. The system will then verify the revised inputs to determine if the information is accurate. After verifying the contents of the document, the system may store the document that was uploaded by the user.

After the user uploads the documents to the system, the system checks to determine if the upload of the documents is complete at 335. The system might review the document that was uploaded or scanned to confirm that the image quality is good. If the image of the document is only partially uploaded, then the upload has not been completed, and the process goes back to 330 to allow the user to upload or scan the documents again. After the documents have been uploaded, the process proceeds again to 335 to verify the upload of the documents has been completed.

After the documents have been uploaded, the process proceeds to 340 in which the system generates an electronic medical badge. The electronic medical badge may include a unique 2D barcode which embeds the location in the system where the verified medical vaccination information is stored, or the verified medical testing information for the user is stored, or alternatively, embeds the verified medical information itself.

After the electronic medical badge has been generated, the system may proceed to 345 to store the electronic medical badge in a database along with the medical vaccine or medical test for which it pertains.

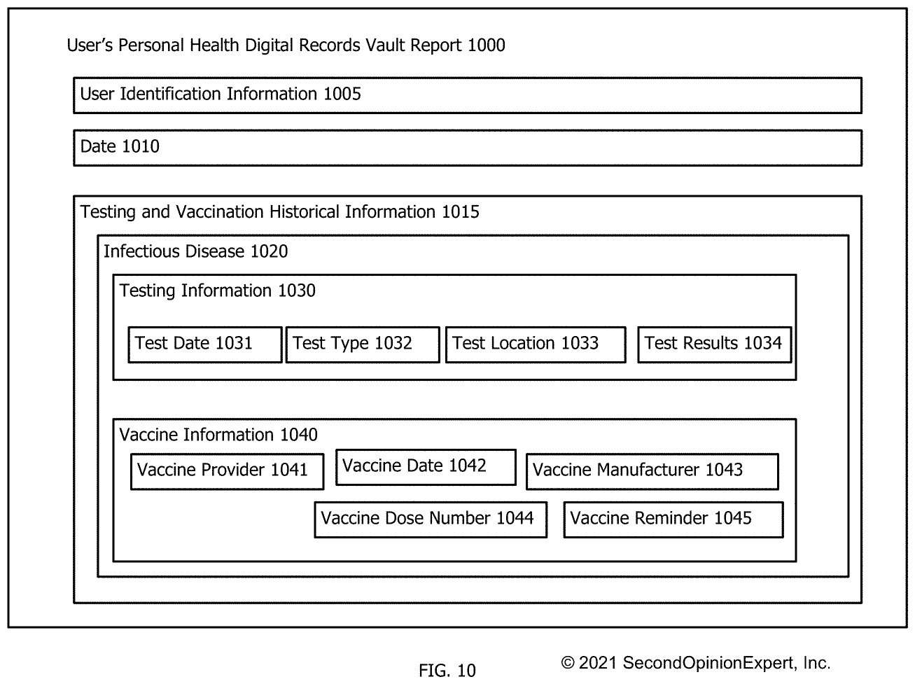

After the electronic medical badge is stored in the system, the system may proceed to 350 to generate a personal health digital records vault report. An example of the report can be seen in FIG. 10. This report includes a list of all the vaccines and testing that the user has undertaken. With respect to the testing information, the report might include the dates of any tests the user has taken, the results of those tests, and the type of test that was taken. In addition, the personal health digital records vault report may include all the vaccines that the user has taken in his lifetime, including any tetanus shots, flu shots, and similar vaccinations, and the date those vaccines were administered. Some vaccines require additional shots over one's lifetime, and therefore this comprehensive information may be useful for users to help remind them when additional vaccine shots are required. For example, a tetanus shot is required once every ten years. By storing all of a user's vaccination information in a personal health digital records vault, then it will be convenient for a user to determine when the next dosage of a certain vaccine might be required. The personal health digital records vault report may include the electronic medical badge which may be a unique 2D barcode for each vaccine or test identified in the report. For example, the user may have an electronic medical badge with a unique 2D barcode stored for the COVID-19 virus, and a user may wish to show another individual this unique 2D barcode so that the other individual may verify the information regarding the vaccine that was administered to the user. By allowing for separate and unique 2D barcodes for each vaccine or test, the user can choose to reveal only the vaccination or testing information that the user wishes to reveal, and nothing more. For example, the user can choose to display the unique 2D barcode for the COVID-19 vaccine, and none of the user's other medical vaccination history.

After the personal health digital records vault report has been generated, then the process moves to 355 to allow the user to view the report. The report will show the user's personal information, and it will also include all the user's medical vaccination and medical testing information.

After the user has viewed the report, the process moves to 360 to determine if the user is able to view the report. If the user is not able to view the report, then the process returns to 355 to make the system view the report. For example, if the user views the report and determines that the 2D barcodes are appearing for some vaccines, but not for all of the vaccines, then the user will request that the system redisplay the report again. If the system is able to view the report, then the process proceeds to 365 to allow the user to download the report. For example, the user may wish to download the report to his smart device so that if he is travelling and he doesn't have any Internet connectivity, then he can still be able to show his vaccination and testing information to the travel authorities as he would have saved the report on his smart device. For example, the user may wish to download the report to his mobile wallet, which may be an application on his smart device. After the user has downloaded the report, the process ends at 395.

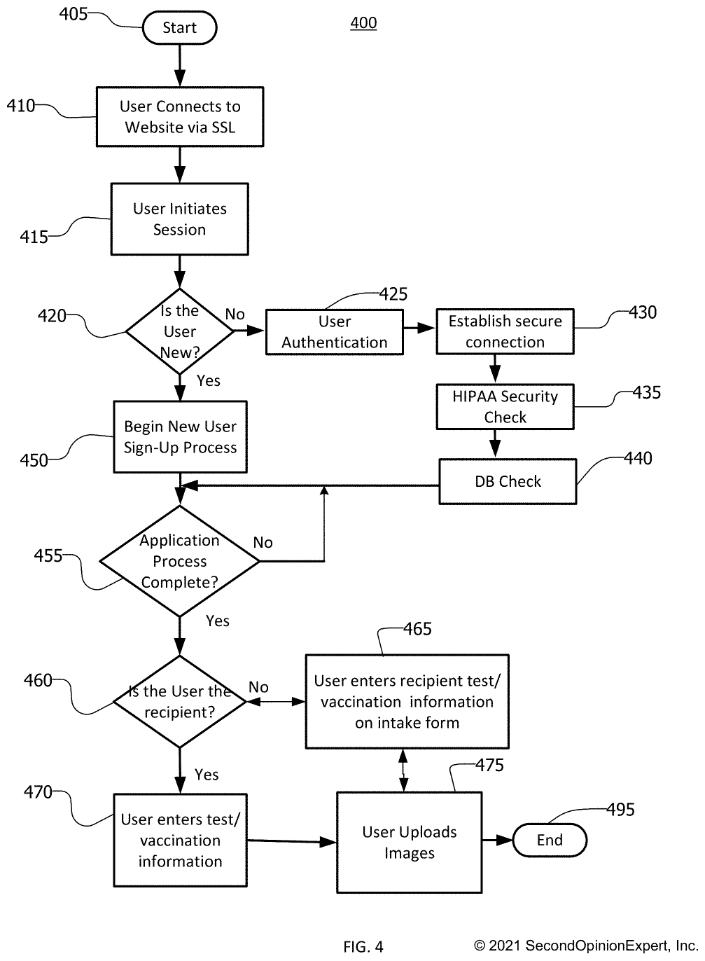

Referring now to FIG. 4, a process 400 for initiating an electronic medical badge is shown. The process 400 may start at 405 and may finish at 495. The process may be started by a user using a user system, such as user system 110 in FIG. 1.

The process 400 may begin with a user accessing the Internet over a secure network connection, such as Secure Sockets Layer (SSL) or Secure HTTP (S-HTTP). At 405, the user connects to the Internet using the user system. The connection needs to be a secure connection as personal information may be exchanged during the process. For example, the user's age, gender, city of residence, credit card information and bank account information may be used to complete the user's login to the system.

At 415, the user initiates a session with the system. The user may initiate the system by opening a Web browser, or by opening an app on a mobile device. At 415, the user may initiate the session by entering the URL for logging into a web server located on the vaccine verification system, such as vaccine verification system 220 in FIG. 2. Alternatively, if the user is using an app on a mobile device, then the session may be initiated as soon as the user is signed in. After the user arrives at the URL, he will have the option of registering as a new user on the website or logging into the website with an account that has already been registered on the website.

At 420, the vaccine verification system determines whether the user is a new user of the vaccine verification system. If so, then the process proceeds to 450, where the user begins the new user process. A user registers with the system by entering personal information, such as his name, birth date and gender. After entering personal information, the user may choose a username and password that is secure and unique to the user. After selecting a username and password, the user may login into the system and start to generate an electronic medical badge. The new user process includes identifying a username and password for the user's account. The username is unique for each user.

If at 420, the user is not a new user, then the user is asked to enter the login information for his account. At 425, the system authenticates the user's login information and determines whether the correct username and password information have been entered into the system.

If the user has entered the correct information at 425, then at 430, a secured connection is established between the user system, such as user system 110 in FIG. 1, and the vaccine verification system, such as vaccine verification system 120 in FIG. 1. The connection must be secured because sensitive information, such as personal and medical information, is being exchanged. The data exchanged over the connection must be encrypted. The secure connection can be either SSL or S-HTTP.

After a secure connection is established, the process proceeds to 435 where a Health Insurance Portability and Accountability Act (HIPAA) security check is performed which is compliant with the HIPAA Security Rule. HIPAA was developed to protect the privacy and security of health information. The HIPAA Security Rule establishes a national set of security standards for protecting certain health information that is held or transferred in electronic form. The Security Rule requires entities to implement safeguards to ensure that electronic health information will remain secure. The Security Rule protects all identifiable health information that an entity creates, receives, maintains, or transmits in electronic form. Therefore at 435, the system confirms that the connection is secure such that any health information that is created in electronic form will remain secure and compliant with the HIPAA Security Rule.

After the HIPAA security check is performed, the process verifies the user's information in the database at 440. In this step, the user registration database, such as user registration database 245 in FIG. 2, may be checked to verify the user's identity and login information.

After the user has logged in, either as an existing user or as a new user, the process proceeds to 455 where it is determined whether the user has completed all the application information. For example, at 455, the user may need to enter information, such as the user's age and the user's gender. If the user has not completed all the application information, the process returns back to 455 and continues until the user has entered all of the information required to complete the application for creating a new account.

At 460, the user is asked whether the user is the recipient who received the vaccine, or if the user is entering the vaccine information for another user. If the user is entering the medical information for himself, then the process proceeds to step 470 where the user enters the testing and vaccination information for himself. The system may ask the user questions regarding the type of test or vaccine the user recently obtained and which the user wants to enter in the system. In addition, the system may ask if the user requires additional doses of the vaccine. Specifically, the system may ask if the user took the first of two doses or if the user has taken both doses of a 2-shot vaccine.

If the user is not the individual who received the vaccine or medical test, then the process proceeds to 465 during which the user will document the recipient's medical information into the system. For example, the user may be a child of a parent who is taking the parent to a vaccination center. The parent may prefer for the user to enter all the information into the system. This step allows for another user to enter medical vaccination or medical testing information, on behalf of another individual, into the system and keep a user's medical testing and vaccination information current.

After the system has completed collecting the vaccination and testing information from the user, the process proceeds to 475 in which the user uploads medical images. These medical images include test results, vaccine dose information and additional vaccine reminders. The documents may also include imaging tests such as X-Rays, Cat Scans, MRI, MRA, Ultra Sound and other scans. After the user uploads the images, the system verifies the contents of the vaccination and testing information. The system may verify the contents of the document by comparing the information with the information stored by the vaccine provider, or by comparing the information with another entity that stores information regarding the user's medical vaccine or medical testing history. After the information has been verified, the process for initiating the generation of an electronic medical badge is complete.

The flow chart has both a start 405 and an end 495, but the process is cyclical in nature.

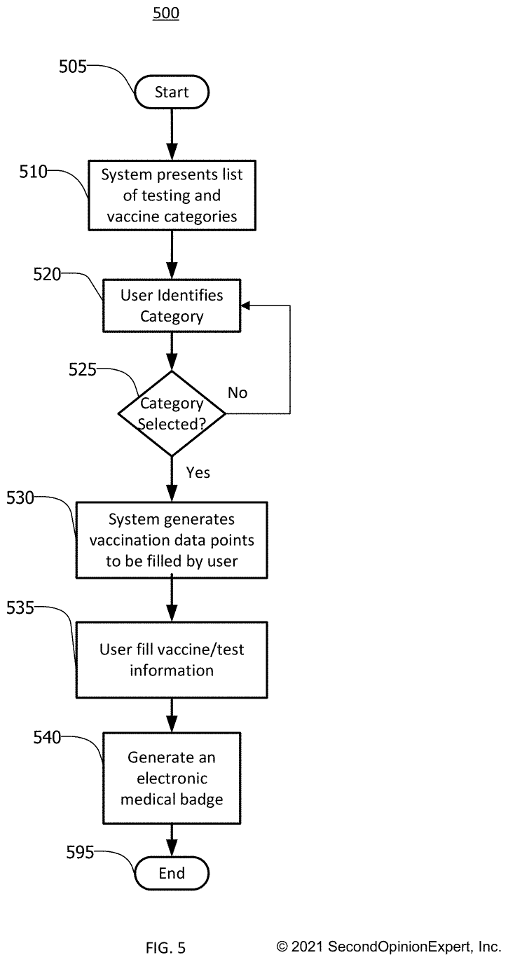

Turning now to FIG. 5, there is shown a flowchart for the process 500 of generating an electronic medical badge. The process 500 starts after the user has logged in securely into the system, and the HIPAA compliance is verified.

At 510, the system presents a list of testing and vaccine categories to the user. The user likely will view the system categories on a display, such as display 216 of FIG. 2. The user will review the categories, and then at 520, the user will select the category that fits the vaccination information or testing information. Examples of categories that might be presented are “COVID-19 Testing”, “COVID-19 Vaccine”, or “Flu Vaccine.”

After the user selects a category, then at 525, the system checks to determine if the user selected a category. If the user has not yet selected a category, then the system presents the categories again to the user. If the user has selected a category, then the system proceeds to 530.

At 530, the system generates the data points and information required for the user to enter the relevant information. For example, if the user selects, “COVID-19 Vaccine”, then the system will present questions for the user to update the manufacturer of the vaccine, the doses and the dates required for the vaccine, the provider who will be administering the vaccine for the user, the location where the vaccine will be administered for the user. At 535, the user will enter the information pertaining to the test or vaccine information for which the user has obtained results. After the user has entered all of the information into the system, the system verifies the information. For example, the system verifies the provider information, and the location information and the accuracy of the vaccine dose information that the user entered. The system may verify the information by comparing the user information, and the accuracy of the vaccine dose information with the information stored in the provider's database. If the information is not the same, the system may prompt the user to enter the correct information.

After the system has collected and verified all of the testing or vaccination information, the system proceeds to 540 in which an electronic medical badge is generated for the user. The electronic medical badge will comprise a 2D code whereby when scanned will reveal the information regarding the vaccine or testing information the user obtained. For example, if the user has obtained the first dose of a 2-dose vaccine, then the 2D barcode will include the vaccine provider, vaccine manufacturer, date the vaccine was administered for the user, and any additional information. The user will be able to display the electronic medical badge on a smart device thereby not requiring any medical documents with him to verify that he is been vaccinated. After the system has generated the electronic medical badge at 540, the process ends at 595. The flow chart has both a start 505 and an end 595, but the process is cyclical in nature.

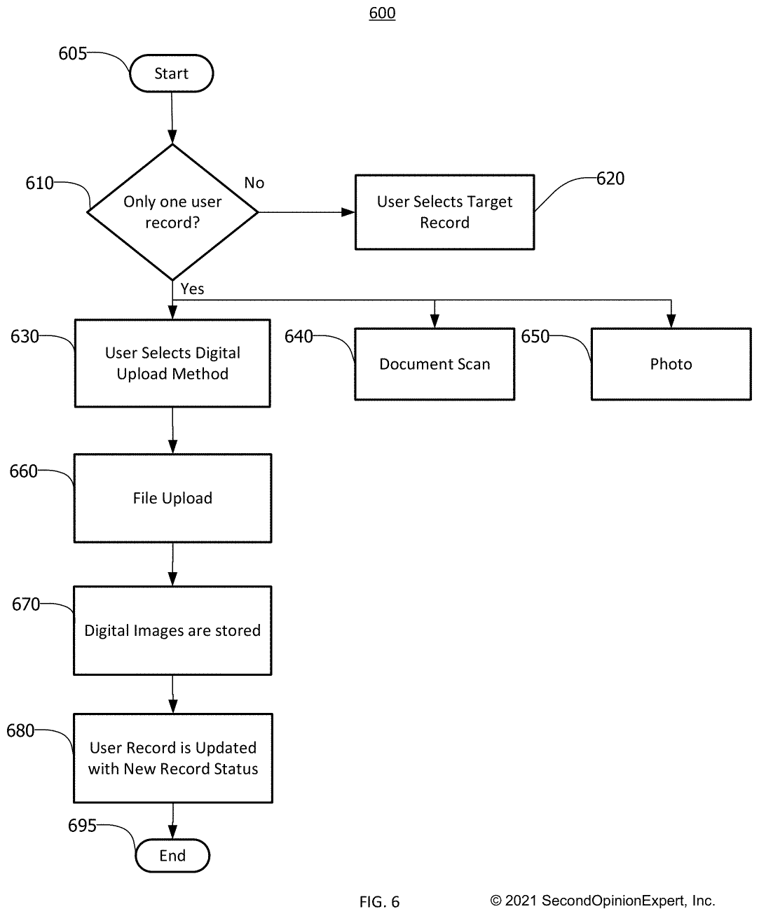

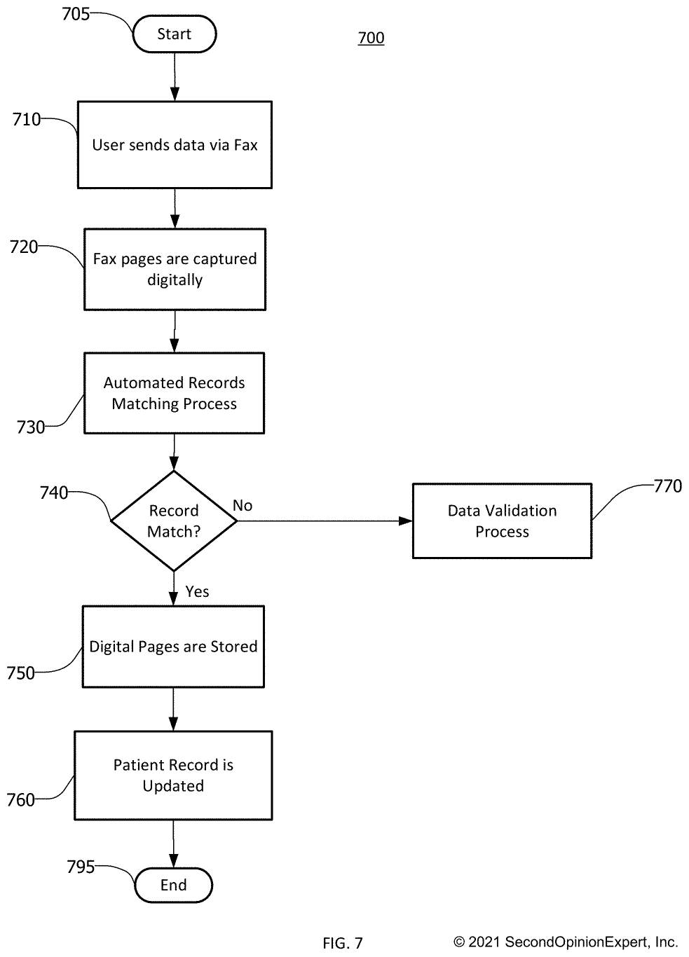

FIGS. 6, and 7 show processes for uploading medical records and transmitting medical records into the system. The processes allow a user to upload a medical record and the system then associates the medical record with a certain user registered with the system. The system allows the user to upload multiple medical records into the system. For example, if a user has obtained multiple doses for a vaccine, and if the user has multiple documents for each dose of the vaccine, then the user will be able to upload the medical documents for each dose of the vaccine.

Turning now to FIG. 6, a flowchart 600 for uploading digital medical records to be used to generate an electronic medical badge is shown. At step 610, the system asks the user whether the user wants to update only one user record. For example, the user may want to update the system to provide information related to a recent medical test that the user took to determine if the user was carrying a particular virus. The user may also wish to update the system to provide information related to a recent vaccine the user obtained in addition to the medical test the user recently took. If the user wishes to update multiple records in the system, such as information related to a particular vaccine the user obtained and information related to a particular test the user obtained, then the system proceeds to 620 to have the user select the record in the system that the user wishes to update. For example, the user will have to select whether the user wishes to update the vaccine information first or the testing information.

After the user selects the record that the user wishes to update, the user selects the digital upload method at 630. The user can also identify whether he wishes the document to be scanned at 640 and whether there is a photo in the document at 650. Once the user selects the options for the upload, then the user may upload the document at 660. The system may verify the contents of the document. The system may verify the contents of the document by comparing the information with the information stored by the vaccine provider, or by comparing the information with another entity that stores information regarding the user's medical vaccine or medical testing history.

After the upload is complete, the digital images are stored in a database at 670, for example the document database 229 as shown in FIG. 2. After the upload is complete, then the user record is updated in the database to identify that documents exist for that record. After the system has updated the database with the medical documents, the process ends at 695. The flow chart has both a start 605 and an end 695, but the process is cyclical in nature.

FIG. 7 shows a flowchart 700 for faxing medical records to be used to generate an electronic medical badge. At 710, the user faxes a medical document to a phone number connected to the system. At 720, the system converts the faxed document data to digital data. At 730, the system reviews the records in the database and determines whether the user for whom the document pertains is in the system. If the user is in the system, then the process proceeds to 740 to see if there is a record match.

If there is not a record match in the database, then the process proceeds to 770 to validate the data and determine to whom this document belongs.

If there is a record match in the database, then the digital copy of the faxed document is stored in the database at 750. At 760, the user's record is updated in the database to reflect that a medical document is saved for that user. The system may verify the contents of the document stored in the system. The system may verify the contents of the document by comparing the information with the information stored by the vaccine provider, or by comparing the information with another entity that stores information regarding the user's medical vaccine or medical testing history.

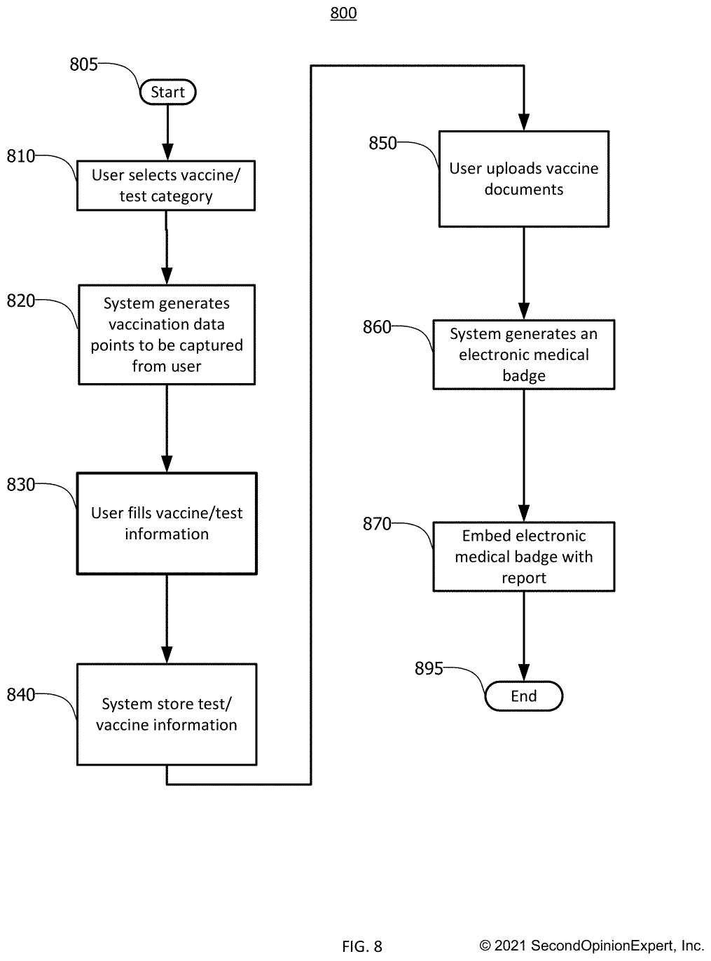

Turning now to FIG. 8, there is shown a flowchart 800 for generating an electronic medical badge and embedding it in a report. At 810, a system selects a vaccine or test category. For example, the user may select “COVID-19 vaccine” or “COVID-19 test”.

After the user selects the vaccine or test category, then at 820, the system generates vaccination data points to be captured from the user. For example, if the user selects “COVID-19 vaccine”, then the system will present inputs for the user to fill such as the vaccine manufacturer, the vaccine provider (if different), the dose number of the vaccine, the location the vaccine was administered, and if any information is required for additional doses to be administered.

At 830, the user fills all of the information in the system relating to the vaccine or test category that the user initially selected in 810. At 840, the system stores the test or vaccine information into the system. For example, the system may store the test or vaccine information into an immunization database in the system, such as immunization database 227 shown in FIG. 2. If the user selects “COVID-19 vaccine” as in the example above, then the system will store the vaccine manufacturer, the vaccine provider, the dose number of the vaccine, the location the vaccine was administered, and any additional information the user entered pertaining to the COVID-19 vaccine that was administered to him.

At 850, the user uploads the vaccine documents to the system. If the user has a physical document with all the relevant vaccine or test information, then the user can use a similar process as shown in FIG. 7 to fax the physical document to be uploaded into the system. Alternatively, the user can use a similar process as shown in FIG. 6 to upload a digital image of the test or vaccine information. After the documents have been uploaded to the system, the system verifies the contents of the documents. After the documents have been verified, these documents may be stored in a database in the system, such as the document database 227 shown in FIG. 2. In addition, the user's personal health digital records digital vault may be updated to include links to the documents that the user just uploaded into the system. This will ensure that all of the user's medical information is stored as part of his personal health digital records vault.

After the documents have been uploaded to the system, the system generates an electronic medical badge at 860. The electronic medical badge may be a 2D barcode which when scanned provides another individual information about the user's vaccination or medical testing information.

At 870, the system embeds the electronic medical badge with the user's report. For example, the system may embed the electronic medical badge with the user's personal health digital records vault report. After the electronic medical badge is embedded in the report, the process ends at 895.

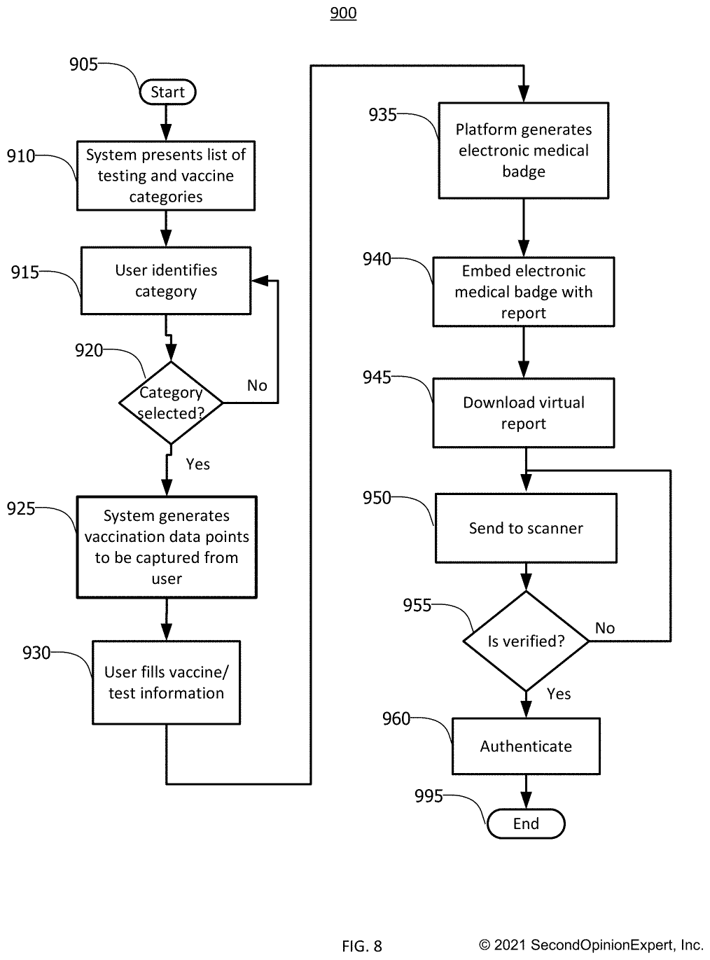

Turning now to FIG. 9, there is shown a flowchart 900 for scanning an electronic medical badge. The process begins at 910 with the system presenting a list of testing and vaccination categories for a user. The process then proceeds to 915 where the user may review the categories and select the category that pertains to the information he wants to enter in the system. For example, if the user recently obtained a COVID-19 antigen test, then he could select the “COVID-19 test” category.

At 920, the system determines if the user has selected a category. If the user has not selected any category, then the system returns to 915 to allow the user to select a category. If the user has selected a category, then the system proceeds to 925 in which the system generates vaccination data points, or medical testing data points, to be captured from the user. For example, if the user selected “COVID-19 test” as the category, then the system may request additional data points such as the type of test the user took, where the user took the test, and the result of the test.

The system then proceeds to 930 in which the user enters or fills in the information in the system. For example, the user may identify that he took a COVID-19 antigen test, the date that he took the test, and the result of the test.

After the user enters all of the information pertaining to the medical vaccination or medical test information, then the system proceeds to 935 and generates an electronic medical badge. The system may generate a unique 2D barcode to embed the relevant medical vaccination or medical test information, or alternatively, the 2D barcode may embed a link in the system where the medical vaccination or medical test information can be retrieved from the system. This electronic medical badge may be displayed on a user's smart device so that another individual may scan the electronic medical badge and cause the system to trigger an inquiry to the system to return and display the information stored in the system on the other user's smart device.

After the electronic medical badge has been generated, the system proceeds to 940 to embed the electronic medical badge with a report, such as a user's personal health digital records vault report. A user may store all of his medical vaccination information or medical testing information in a personal health digital records vault which may be stored on his smart device. In this step, the system may embed the electronic medical badge information in the user's personal health digital records vault, and whenever a user generates a personal health digital records vault report, the electronic medical badge will be embedded in the report.

After the electronic medical badge is embedded in the report, the system proceeds to 945 in which the user's personal health digital records vault report is downloaded to a device. The user may download the report to his device so as to enable him to select the vaccine information and related electronic medical badge information to display to another individual.

After the report has been downloaded, the system may proceed to 950 in which another user may be allowed to use a 2D barcode scanner, or a camera on their smart device, to scan the electronic medical badge shown on the user's smart device.

After the other user has scanned the electronic medical badge, the system determines at 955 if the electronic medical badge has been verified. If the system is unable to verify the electronic medical badge, then the system returns to 950 to try to obtain another scan of the electronic medical badge.

If the system is able to verify the electronic medical badge, then the system proceeds to 960 to authenticate the information in the electronic medical badge. In this step, the other user may be able to view the contents of the medical vaccination information, or the medical testing information of the other user.

FIG. 10 is an example of a user's personal health digital records vault report. The report can have various formats, and this is shown just as an example. The report 1000 has user identification information 1005. This may include information regarding the user's name, gender and age.

The report 1000 also includes the date of the report 1010. Each time a report is generated, the date will be updated with the current date so that anyone viewing it will be able to verify it is the most current information.

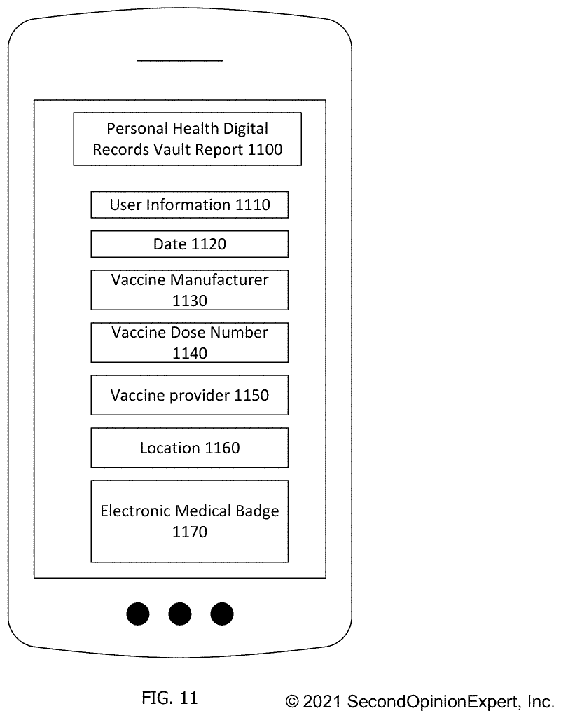

The report 1000 also includes testing and vaccination 1015 information. This portion of the report may include all the user's medical testing and medical vaccination history for the user. Alternatively, the report may include only a portion of the user's medical testing and medical vaccination information. An example of this is shown in FIG. 11 and will be discussed further below.

The testing and vaccination 1015 information may comprise an infection disease 1020 record. This infectious disease 1020 record may identify a particular infectious disease which contains information for the user. For example, the infectious disease 1020 record may be a record for measles. Within the infectious disease 1020 record, there may be testing information 1030, and vaccination information 1040.

The testing information 1030 may include the test date 1031, test type 1032, test location 1033, and test results 1034. For example, if a user took a COVID-19 test, then the personal health digital records vault report may include the test date, the test type, test location and the test results in the report. The testing information 1030 may include additional information, such as the document showing the test results. The testing information 1030 may also include additional information as well pertaining to the test taken.

The vaccine information 1040 may include the vaccine provider 1041, vaccine date 1042, vaccine manufacturer 1043, vaccine dose number 1044, and vaccine reminder 1045. For example, if a user obtained a COVID-19 vaccine, then the personal health digital records vault report may include the vaccine provider, such as the entity the administered the vaccine. If the user received the vaccine at a hospital, then the vaccine provider will be the name of the hospital. In addition, the vaccine information 1040 may include the date the vaccine was administered, as shown in 1042. In addition, the vaccine information 1040 may include name of the manufacturer of the vaccine. For example, if a user received a COVID-19 vaccine, then the vaccine manufacturer may identify Pfizer or Moderna as the name of the manufacturer. The vaccine information 1040 may also include the vaccine dose number 1044. Some vaccines, such as the vaccines currently available for COVID-19, require at least two doses. The vaccine dose number 1044 will identify which dose the user has received. If multiple doses are required of a vaccine, then the report may also include a vaccine reminder 1045 which may identify a date when the user needs to register for the next dose of the vaccine. The vaccine information 1040 may also include additional information, such as a digital image of the document showing that the vaccine was administered to the user. Additional information regarding the vaccine may also be included in the vaccine information 1040.

The personal health digital records vault report may include multiple records, similar to infectious disease 1020 record, for each vaccine and test that the user has received in his lifetime. The report may be a comprehensive personal health digital records vault report which lists all the user's vaccines and tests to date.

Referring now to FIG. 11, there is shown a portion of a personal health digital records vault report 1100 on a display of a smart device. The personal health digital records vault report 1100 may include user information 1110. The user information may identify the user's name, the user's gender, and the user's date of birth. The report may also include the date 1120 of the report. Another individual may use the date of the report to confirm that it is the most current information. The report may also include the vaccine manufacturer 1130. It is helpful to include the vaccine manufacturer, as different manufacturer's vaccines have different doses and different times for which additional doses must be administered. The report may also include the vaccine dose number 1140. For those vaccines that include multiple doses, it is helpful to know how many doses an individual has received. Some facilities may only require that one dose of a vaccine has been administered for a user to use the facilities, and other facility owners may require that a user has obtained all doses of a vaccine before using the facilities. The report may also include information regarding the vaccine provider 1150. The report may also include the location 1160 that the user obtained the vaccine. In addition, the report may include the electronic medical badge 1170. Another user may be able to scan the electronic medical badge to be able to verify the vaccination information or testing information of the user.

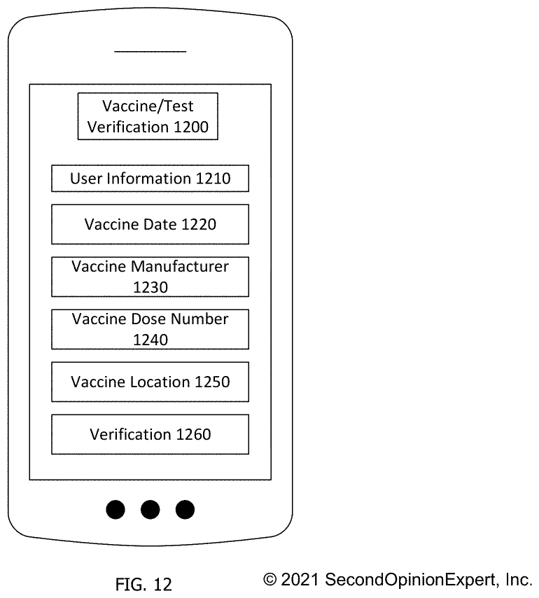

Referring now to FIG. 12, there is shown an example of another user's display after scanning the user's electronic medical badge. Another user may be presented with a user's personal health digital records vault report as the one shown in FIG. 11. The other user may use his smart device, or scanner, to scan the electronic medical badge of the user showing his display. After scanning the electronic medical badge, the vaccine or test verification 1200 display will appear on the user's smart device.

The vaccine or test verification display may include the user information 1210. This may include the user's name and the user's gender. If the user does not wish to reveal his date of birth, then he can choose not to reveal that to the other user. The vaccine or test verification display may also include the vaccine date 1220 which will state the date the vaccine was administered to the user. The vaccine or test verification display may also include the vaccine manufacturer 1230. It may also include the vaccine dose number 1240. In addition, it may also include the vaccine location 1250. Finally, it may also include verification 1260. The verification 1260 is a symbol confirming that the information displayed on the screen has been verified as accurate. The other user will be able to view the verification information and determine the vaccine information or medical test information for the user.

FIGS. 10, 11 and 12 are just included to provide as an example. The actual displays may take many different forms and does not need to include all of the shown elements.

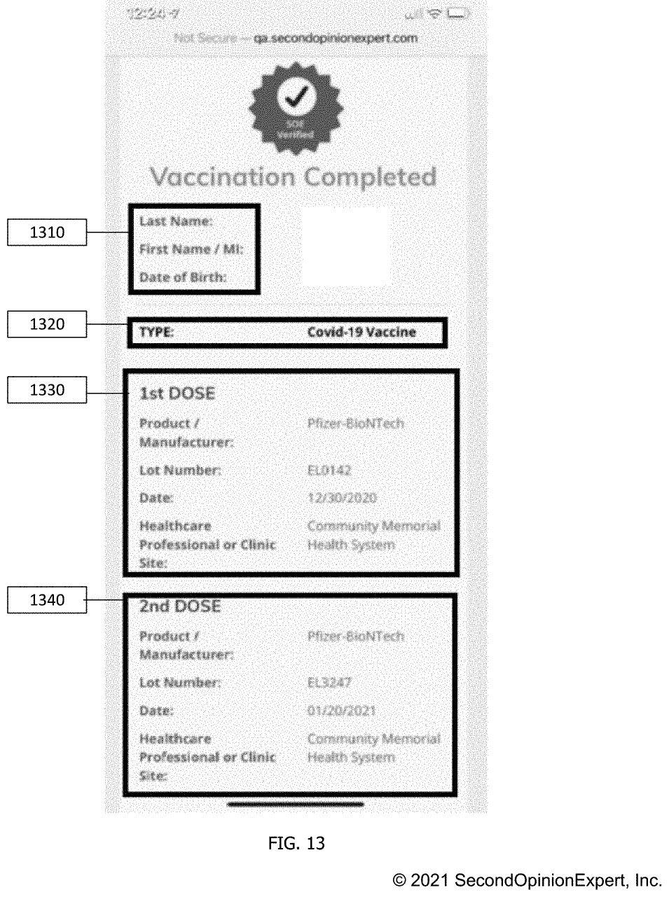

FIG. 13 is an example of a portion of a user's personal health digital records vault report that may be displayed on a user's electronic device showing a user's vaccination information. FIG. 13 includes the user information 1310. For example, this might include the user's first and last name, and their date of birth. FIG. 13 also includes the type 1320 of vaccine that was administered. In this example, the type is the Covid-19 Vaccine. FIG. 13 also includes information regarding the two vaccine doses. In this example, the display includes the first vaccine dose information 1330 and the second vaccine dose information 1340. The vaccine dose information includes the manufacturer of the vaccine, in this case, Pfizer. It also includes the date that each dose was administered, and it also includes the location that the vaccine was administered. It also identifies the lot number.

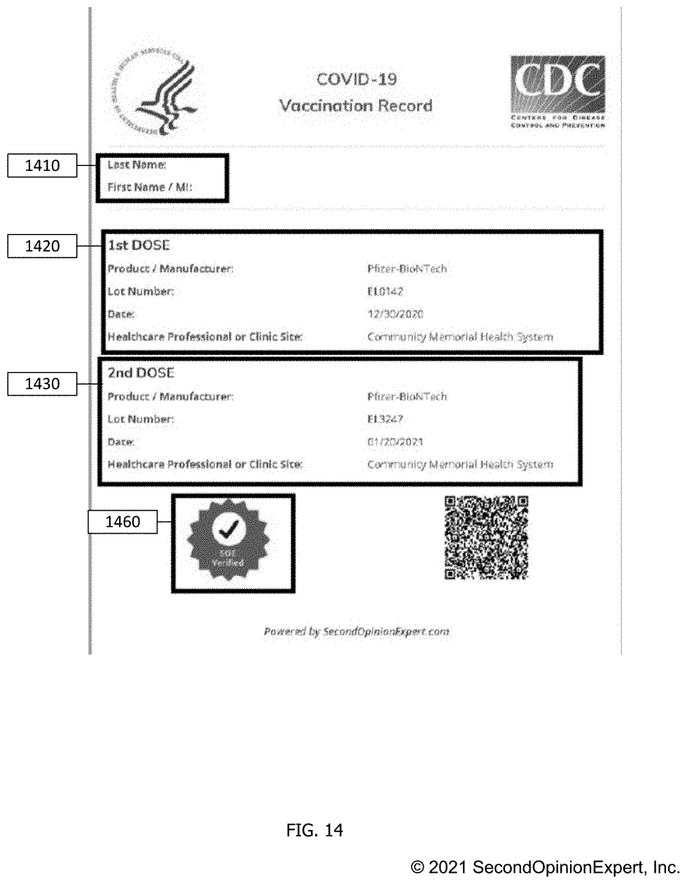

FIG. 14 is another example of another user's display after scanning the user's electronic medical badge. FIG. 14 includes the user information 1410. For example, this might include the user's first and last name. FIG. 14 also includes information regarding the two vaccine doses. In this example, the display includes the first vaccine dose information 1420 and the second vaccine dose information 1430. The vaccine dose information includes the manufacturer of the vaccine, in this case, Pfizer. It also includes the date that each dose was administered, and it also includes the location that the vaccine was administered. It also identifies the lot number. FIG. 14 also includes an identification 1460 that the vaccine information that is displayed on the screen was verified.

Closing Comments

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. With regard to flowcharts, additional and fewer steps may be taken, and the steps as shown may be combined or further refined to achieve the methods described herein. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

Within this description, the term “engine” means a collection of hardware, which may be augmented by firmware and/or software that performs the described functions. An engine may typically be designed using a hardware description language (HDL) that defines the engine primarily in functional terms. The HDL design may be verified using an HDL simulation tool. The verified HDL design may then be converted into a gate netlist or other physical description of the engine in a process commonly termed “synthesis”. The synthesis may be performed automatically using a synthesis tool. The gate netlist or other physical description may be further converted into programming code for implementing the engine in a programmable device such as a field programmable gate array (FPGA), a programmable logic device (PLD), or a programmable logic arrays (PLA). The gate netlist or other physical description may be converted into process instructions and masks for fabricating the engine within an application specific integrated circuit (ASIC).

Within this description, the term “unit” also means a collection of hardware, firmware, and/or software, which may be on a larger scale than an “engine”. For example, a unit may contain multiple engines, some of which may perform similar functions in parallel. The terms “engine” and “unit” do not imply any physical separation or demarcation. All or portions of one or more units and/or engines may be collocated on a common card, or within a common FPGA, ASIC, or other circuit device.

Although shown implemented in a personal computer, the processes and apparatus may be implemented with any computing device. A computing device as used herein refers to any device with a processor, memory and a storage device that may execute instructions including, but not limited to, personal computers, server computers, computing tablets, laptop computers, smart devices including smart phones and smart watches, set top boxes, video game systems, personal video recorders, telephones, personal digital assistants (PDAs), and portable computers. These computing devices may run an operating system, including, for example, variations of the Linux, Microsoft Windows, Symbian, and Apple Mac operating systems.

The techniques may be implemented with machine readable storage media in a storage device included with or otherwise coupled or attached to a computing device. That is, the software may be stored in electronic, machine readable media. These storage media include, for example, magnetic media such as hard disks, optical media such as compact disks (CD-ROM and CD-RW) and digital versatile disks (DVD and DVD±RW); flash memory cards; and other storage media. As used herein, a storage device is a device that allows for reading and/or writing to a storage medium. Storage devices include hard disk drives, DVD drives, flash memory devices, and others.

It is claimed:

1. A method for generating an electronic medical badge comprising:
a user interacting directly with a computing device to identify a medical vaccine that the user recently obtained;
the computing device receiving a document from the user, the document comprising information regarding the medical vaccine information the user recently obtained, including a vaccine manufacturer, a vaccine provider, a vaccine location; a vaccine date, and a vaccine dose;
the computing device extracting the medical vaccine information from the document, including the vaccine manufacturer, the vaccine provider, the vaccine location;
the computing device storing the document in a database;
the computing device generating an electronic medical badge comprising a 2D barcode that may be displayed on a smart device, wherein the 2D barcode is embedded with the user's medical vaccine information wherein the user's medical vaccine information has been verified.

2. The method of claim 1 wherein the computing device embeds the electronic medical badge in a user's personal health digital records vault which may be stored on a user's smart device.

3. The method of claim 2 wherein the computing device generates a personal health digital records vault report, wherein the user's electronic medical badge is included in the personal health digital records vault report.

4. The method of claim 3 wherein the user downloads the personal health digital records vault report on to the smart device.

5. The method of claim 4 wherein the user displays the personal health digital records vault report on the smart device.

6. The method of claim 5 wherein the personal health digital records vault report includes the vaccine date, the vaccine manufacturer, the vaccine dose number, and the electronic medical badge.

7. The method of claim 6 wherein a second computing device scans the electronic medical badge.

8. The method of claim 7 wherein the second computing device displays the vaccine date, the vaccine manufacturer, and the vaccine dose number of the user.

9. The method of claim 3 wherein the personal health digital records vault report includes a digital image of the document.

10. A system for generating an electronic medical badge comprising a storage medium storing a program having instructions which when executed by a processor will cause the processor to:
identify a medical vaccine that the user recently obtained;
receive a document from the user, the document comprising information regarding the medical vaccine information the user recently obtained, including a vaccine manufacturer, a vaccine provider, a vaccine location; a vaccine date, and a vaccine dose;
extract the medical vaccine information from the document, including the vaccine manufacturer, the vaccine provider, the vaccine location;
store the document in a database;
generate an electronic medical badge comprising a 2D barcode that may be displayed on a smart device, wherein the 2D barcode is embedded with the user's medical vaccine information wherein the user's medical vaccine information has been verified.

11. The system of claim 10 further comprising embedding the electronic medical badge in a user's personal health digital records vault which may be stored on a user's smart device.

12. The system of claim 11 further comprising generating a personal health digital records vault report, wherein the user's electronic medical badge is included in the personal health digital records vault report.

13. The system of claim 12 wherein the user downloads the personal health digital records vault report on to a user's smart device.

14. The system of claim 13 wherein the user displays the personal health digital records vault report on the smart device.

15. The system of claim 14 wherein the personal health digital records vault report includes the vaccine date, the vaccine manufacturer, the vaccine dose number, and the electronic medical badge.

16. The system of claim 15 wherein a second computing device scans the electronic medical badge.

17. The system of claim 16 wherein the second computing device displays the vaccine date, the vaccine manufacturer, and the vaccine dose number of the user.

18. The system of claim 13 wherein the personal health digital records vault report includes a digital image of the document.

19. The system of claim 12 wherein the personal health digital records vault report lists all of the vaccines the user has obtained in his lifetime.
